(19) United States
(12) Patent Application Publication
(10) Pub. No.: US 2026/0232478 A1
(43) Pub. Date: Aug. 13, 2026

(54) PERISTOMAL SKIN BARRIER SENSOR

(71) Applicant: HOLLISTER INCORPORATED, Libertyville, IL (US)

(72) Inventors: Gracia Cosenza, Evanston, IL (US); Huma Zulfiqar Khan, Corona, CA (US); Aymen Lamsahel, Chicago, IL (US); Jonathan Li Chen, Evanston, IL (US); Madison Russo, Evanston, IL (US); Wala Siddig, Iowa City, IA (US); Ross Wojcik, Overland Park, KS (US); Adrian P. Defante, Chicago, IL (US); Mark W. Jockel, Vernon Hills, IL (US)

(73) Assignee: Hollister Incorporated, Libertyville, IL (US)

(21) Appl. No.: 19/156,262

(22) PCT Filed: Mar. 20, 2024
(86) PCT No.: PCT/US2024/020655

§ 371 Date: Aug. 13, 2025

Related U.S. Application Data
(60) Provisional application No. 63/491,951, filed on Mar. 23, 2023.

(51) Int. Cl. A61F 5/44; A61F 5/443

(52) U.S. Cl. CPC A61F 5/4404; A61F 5/443

(57) ABSTRACT

A peristomal skin barrier sensor includes a flexible substrate, a flexible angle sensor strip positioned on the substrate, a. series of teeth-like projections positioned on the flexible angle sensor strip and conductors mounted on a portion of at least son of teeth-like projections of the series of teeth-like projections. The flexible angle sensor and the conductors are in electrical communication with a processor, and wherein a location of a bend in the sensor is identified via electrical signals from the conductors and an angle of the bend in the sensor is identified via. electrical signals from the flexible angle sensor. A. peristomal skin barrier having a. skin barrier sensor is also disclosed.

### BACKGROUND

The present disclosure is related to a peristomal skin barrier sensor. More particularly, the present disclosure is directed to a peristomal skin barrier to monitor the mechanical integrity and deformation of an ostomy appliance adhesive skin barrier throughout use.

An ostomy is a surgical procedure that reroutes parts of the digestive or urinary system to a stoma—an opening in the abdomen through which waste can pass and be collected in a pouch. An adhesive barrier attaches the ostomy pouch to the skin surrounding the stoma to enable waste to be consistently collected in the pouch. During use, the adhesive barrier can move and detach from the skin, contributing to a leakage of bodily waste and/or a complete separation of the pouch from the abdomen. In an ideal case, the adhesive barrier and skin would move in unison, but often the barrier detaches from the skin due to the magnitude and direction of the shear and normal stresses that are induced by everyday activities. This detachment can depend on the intensity, frequency, and longevity of activity.

Currently there are no methods to measure the movement of the adhesive barrier. An understanding is needed of the changes the barrier undergoes relative skin movement in order to create adhesives that better compliment the skin and skin movement in use.

Accordingly, there is a need for a peristomal skin barrier sensor. Such a sensor monitors contact of the barrier and the peristomal skin and is readily adhered to the flexible barrier (substrate). Desirably, such a monitor functions to understand the location and angle of bend of the barrier, as well as the stresses experienced at these locations.

### BRIEF SUMMARY

In one aspect, a peristomal skin barrier sensor includes a flexible substrate, a flexible angle sensor strip positioned on the substrate, a series of teeth-like projections positioned on the flexible angle sensor strip and conductors mounted on a portion of at least some of teeth-like projections of the series of teeth-like projections. The flexible angle sensor and the conductors are in electrical communication with a processor, and a location of a bend in the sensor is identified via electrical signals from the conductors and an angle of the bend in the sensor is identified via electrical signals from the flexible angle sensor. The series of teeth-like projections and the conductors can form a normally open sensor or a normally closed sensor.

In embodiments, the teeth-like projections can be, for example, trapezoidal shaped teeth and the conductors are mounted on an angled surface of the trapezoidal shaped teeth. Sides of the trapezoidal shaped teeth-like projections extend upwardly at an angle relative to horizontal axis through the teeth. One suitable angle is about 20 degrees. The teeth can have an open interior.

The teeth-like projections can have a first upwardly extending surface at a first angle relative to horizontal axis through the teeth-like projections and a second upwardly extending surface extending from the first upwardly extending surface at a second angle relative to the horizontal axis. In such an embodiment, the first angle is less than the second angle. The first angle can be about 5 degrees to about 10 degrees and the second angle can be about 10 degrees to about 20 degrees. In the multi-angle configuration, a first conductor is positioned on the first surface and a second different conductor is positioned on the second surface.

In embodiments, the sensor includes a plurality of series of teeth-like projections, and the conductors on each of the plurality of series of teeth-like projections is electrically isolated from the conductors on the others of the plurality of series of teeth-like projections.

In an aspect, the series of teeth-like projections and the conductors can form a normally closed sensor The teeth-like projections can have a round profile.

In an aspect, a peristomal skin barrier sensor includes a skin barrier, wherein the skin barrier surrounds a stoma that emits ostomy dejecta and a skin barrier sensor configured to a sense movement of the skin barrier, the skin barrier sensor having flexible substrate, a flexible angle sensor strip positioned on the substrate, a series of teeth-like projections positioned on the flexible angle sensor strip, and conductors mounted on a portion of at least some of teeth-like projections of the series of teeth-like projections. The flexible angle sensor and the conductors are in electrical communication with a processor, and wherein a location of a bend in the sensor is identified via electrical signals from the conductors and an angle of the bend in the sensor is identified via electrical signals from the flexible angle sensor.

Other aspects and advantages will become more apparent from the following detailed description when taken in conjunction with the accompanying drawings. The foregoing general description and the following detailed description are examples only and are not restrictive of the present disclosure.

### BRIEF DESCRIPTION OF THE DRAWINGS

The benefits and advantages of the present embodiments will become more readily apparent to those of ordinary skill in the relevant art after reviewing the following detailed description and accompanying drawings, wherein:

FIG. 1 illustrates a user applying an ostomy pouch to a pre-applied barrier;

FIG. 2 illustrates one type of ostomy barrier;

FIGS. 3A and 3B illustrate the front and rear of another type of ostomy barrier;

FIGS. 4A and 4B illustrate the front and rear of still another type of ostomy barrier;

FIGS. 5A-5C illustrate adhesive barrier movement over a three day period

FIGS. 6A and 6B illustrate a stoma with minor skin deformation (FIG. 6A) and a stoma with major skin deformation (FIG. 6B);

FIG. 7 is a functional tree diagram for a peristomal skin barrier sensor;

FIG. 8 is a functional flow diagram for a peristomal skin barrier sensor;

FIGS. 9A and 9B illustrate a series of normally open (NO) upstanding teeth-like contact elements in which, in FIG. 9A the elements are in a fully open state and in which, in FIG. 9B a portion of the contact elements are in a closed state, the elements being trapezoidal element;

FIGS. 10A and 10B illustrate a series of normally closed (NO) upstanding teeth-like contact elements in which, in FIG. 10A the elements are in a fully closed state and in which, in FIG. 10B a portion of the contact elements are in an open state, the elements being rectangular elements;

FIG. 11 illustrates a prototype sensor arrangement in accordance with an embodiment;

FIG. 12 is an embodiment of an angle sensor strip sandwiched between a normally open toothed sensor strip and a normally closed toothed sensor strip;

FIG. 13 illustrates an embodiment of a toothed strip that has seven sensing regions (the spaces between teeth) in which each region has three angle sensing locations;

FIGS. 14-17 illustrate various embodiments of teeth;

FIG. 18 illustrates an embodiment in which a normally open sensor has two sensing regions with angle locations in two axes;

FIG. 19 illustrates a normally closed sensor with three sensing regions;

FIGS. 20A, 20B and 21 illustrates a cantilever model for a chained resistive flex sensor used to predict curvature;

FIG. 22 is a diagram for estimating the radius of the flex sensor relative to a neutral axis;

FIGS. 23A-23D show flex sensors chained end-to-end for more complex predictive modelling;

FIG. 24 illustrates capacitor stretch as the sensor bend stretches;

FIG. 25 shows the sensor output supplementary angle to the angle at which it is bent;

FIG. 26 illustrates an embodiment of a sensor that includes capacitive sensing technology in combination with a BendLabs sensor to detect the location of bends;

FIG. 27 shows the construction of the sensor of FIG. 26;

FIG. 28 is a circuit diagram showing the sensor as connected to a board;

FIG. 29 shows the electrical architecture of an embodiment of the peristomal skin sensor;

FIG. 30 shows the product architecture of the embodiment of the peristomal skin sensor of FIG. 29;

FIG. 31 is a workflow diagram showing how the data collected was used to create a predictive model;

FIG. 32 illustrates the method that was used to find the corresponding displacement to an angle and distance from the end of the sensor;

FIG. 33 shows a model in which there was a fixed relationship on one of the end of the sensor, a fixed relationship along the part of the sensor that was not bending and a prescribed displacement along the other edge of the sensor;

FIGS. 34A and 34B are sample plots of FEA results showing the corresponding stress and strain plots, in which FIG. 34A shows the FEA results for stress and FIG. 34B shows the FEA results for strain;

FIGS. 35A and 35B show the FEA results in a static heat map model (FIG. 35A) and a dynamic heat map model (FIG. 35B); and

FIG. 36 illustrates various angle test fixtures created for testing the peristomal skin sensor.

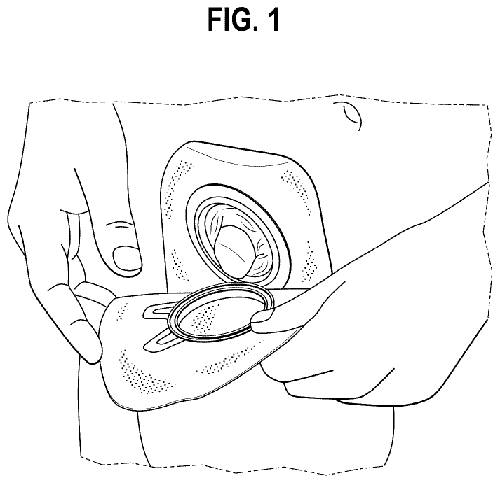

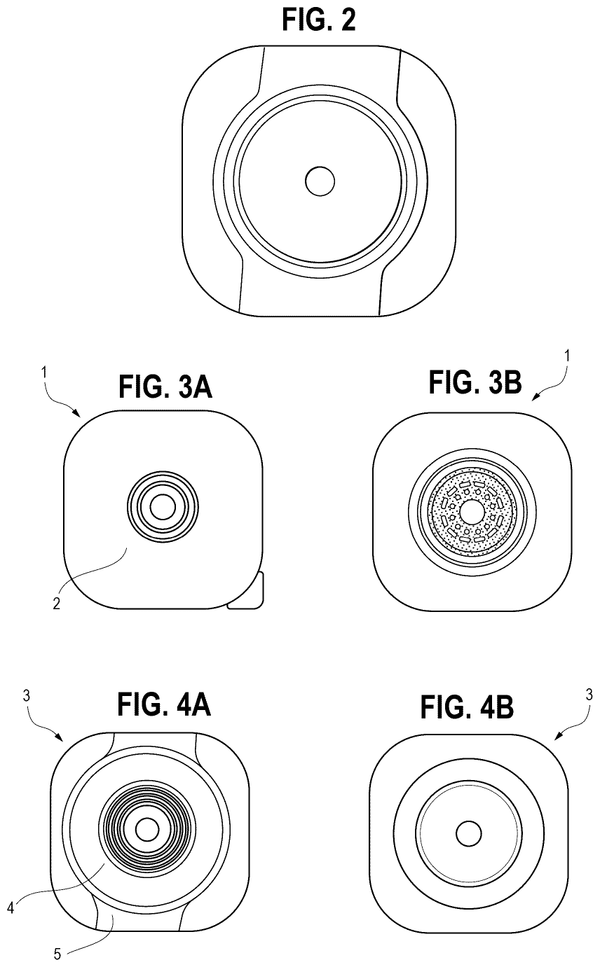

### DETAILED DESCRIPTION

While the present disclosure is susceptible of embodiment in various forms, there is shown in the drawings and will hereinafter be described presently preferred embodiments with the understanding that the present disclosure is to be considered an exemplification and is not intended to limit the disclosure to the specific embodiments illustrated.

An adhesive barrier is used to attach the ostomy pouch to the skin around the stoma (the peristomal skin) to enable waste to be directed into the pouch without leakage. There are different types of adhesive barriers. Referring briefly to FIGS. 3A and 3B illustrate a barrier 1 in which the entirety or nearly the entirety of the rear 2 (FIG. 3A) of the barrier (that side that is attached to the peristomal skin) is made from a hydrocolloid adhesive. FIGS. 4A and 4B illustrate a barrier 3 in which the hydrocolloid material 4 is located the center of the barrier (the central circle in FIG. 4A) and a more band-aid-like tape material 5 surrounds the edges.

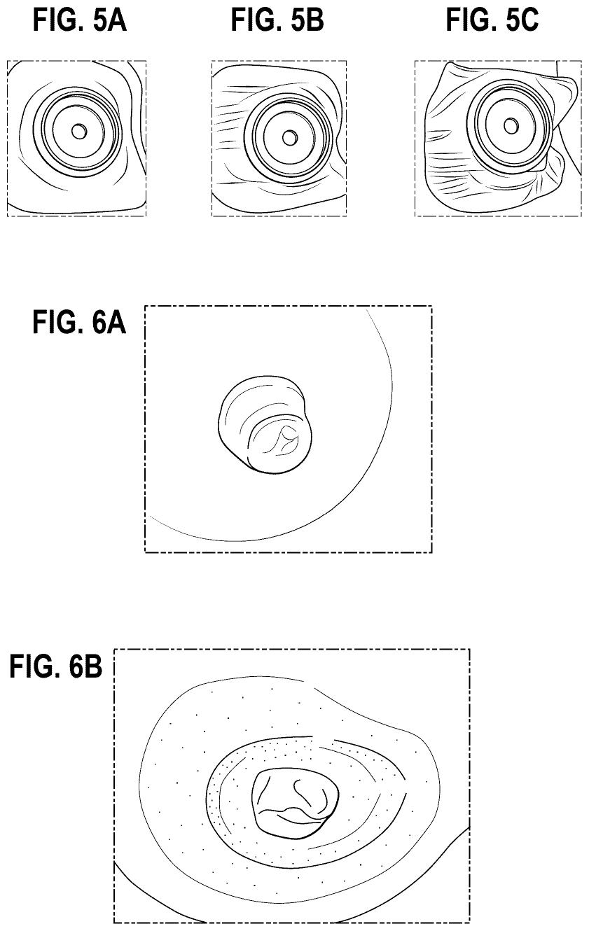

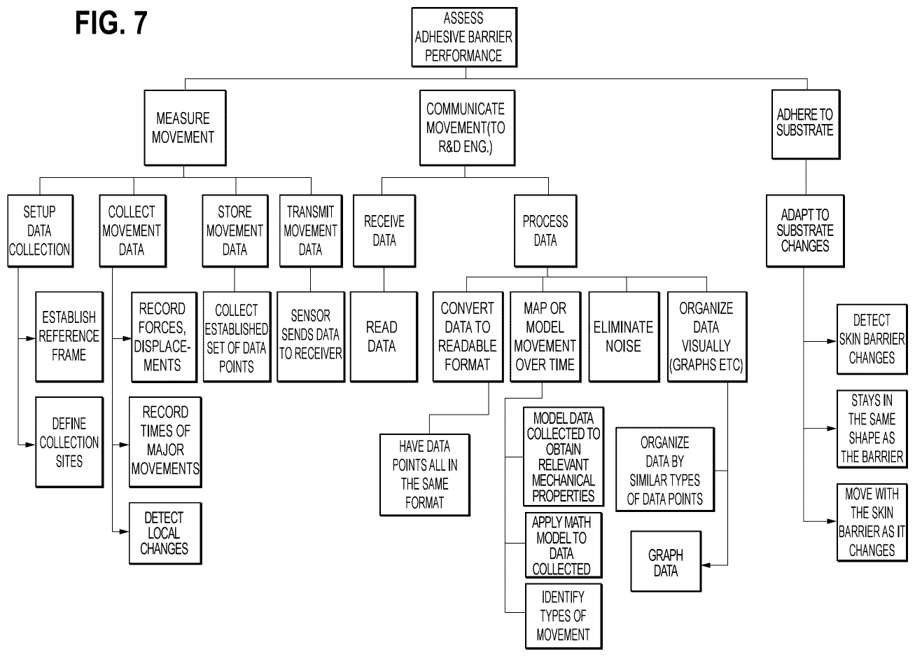

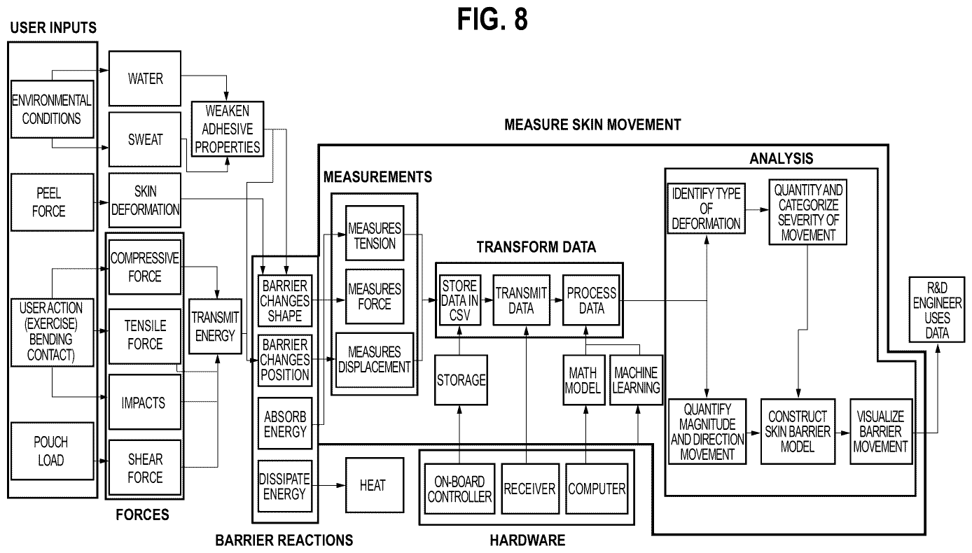

It has been observed that adhesive barriers often move and can detach from the skin, contributing to a leakage of bodily waste and/or complete separation of the pouch from the abdomen. The progression of the movement of the adhesive barrier throughout a three day period was recorded and is shown in FIGS. 5A through 5C. This movement could also cause deformation, irritation, and redness in the skin surrounding the stoma, as shown in FIGS. 6A and 6B.

In aspects, a peristomal skin barrier sensor is a flexible apparatus that detects the location and the angle of bend, measures the deformations that the barrier experiences and tracks its movement on the body over time throughout different activities. As the barrier experiences various types of forces, the sensor facilitates observation of the different types of barrier deformation and the effect that these forces have on the adhesive.

Certain desirable functional characteristics were outlined for such a sensor to assess the performance of an adhesive barrier. Three principal categories of characteristics for such a sensor are to: (1) measure movement; (2) communicate movement data; and (3) adhere to the substrate. Within the three principal areas there are further specific characteristics. Under measure movement, there are (a) set up data collection; (b) collect movement data; (c) store movement data; and (d) transmit movement data. Under communicate movement data, the characteristics are: (a) receive data; and (b) process data. And under adhere to substrate, the characteristic is to: (a) adhere to substrate changes.

As such, the sensor device should securely attach to a surface, adapt to movement of the surface, and be durable and safe to use. The sensor device should measure deformation, collect data over time and collect data at multiple locations on surface. The raw data collected should be accurate and precise and the processed data should be accurate and precise. The data should be obtained in a form of mechanical properties and can be processed into relevant measurements for researchers to evaluate adhesive performance.

To achieve these characteristics, the sensor device should be a wearable device. The Young's modulus value for sensor should be similar to that of the adhesive barrier and/or skin as determined in a tensile test, withstands cyclic fatigue testing, and is reusable for multiple trials. Preferably, such a sensor measures stretching in % strain, measures bending stress in Pa and produces data consistent with theoretical predictions when deformed around known curvatures. Sampling frequency during data collection period should be high as should be the minimum number of data collection sites per unit area (e.g., square centimeter). The data should not significantly differ between trials/tests with the same input and conditions. Deformation data should be captured in displacement, strain, force, and radius of curvature.

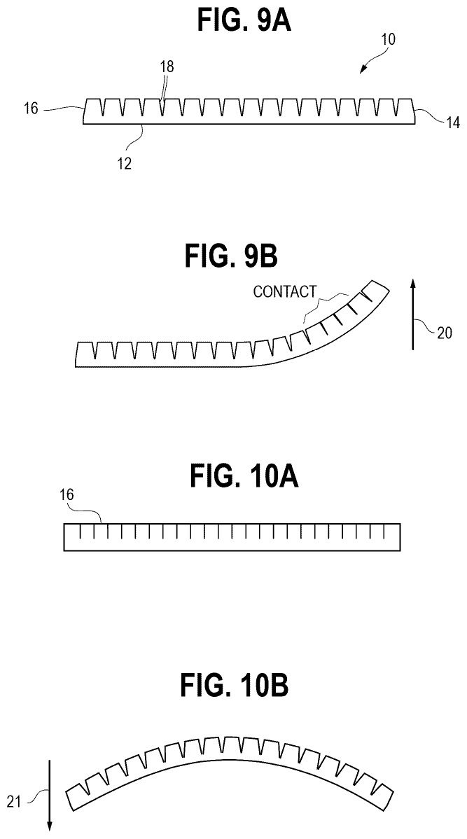

FIGS. 9A and 9B illustrate an embodiment of a peristomal skin barrier sensor 10. The illustrated sensor 10 is a flexible member having a base or substrate 12, an angle sensor strip 14, and plurality of upstanding teeth-like elements 16. In the illustrated embodiment of FIGS. 9A and 9B, the teeth 16 are trapezoidal shaped; in the embodiment illustrated in FIGS. 10A and 10B. the teeth 16 are rectangular. The base 12 can be formed from, for example, a silicone or like flexible material.

One or more angle sensor strips 14, such as flexible soft sensors, such as Bend Labs' flexible 1-axis or 2-axis soft sensors (referred to herein as the angle sensor strip) can be mounted to the base 12. The angle sensor strip 14 can be used to determine the extent or angle to which the sensor strip 14 is bent. The angle sensor strip 14 measures a highly accurate and drift free angular displacement in a soft form factor. The angle sensor strip 14 is made using layered medical grade silicone elastomers doped with conductive and nonconductive fillers, so that they have similar mechanical properties and operating temperatures to other silicone elastomer products. Angle sensor strips 14 measure angular displacement via a differential capacitance measurement, such that common mode signals such as temperature fluctuations, strain and noise are rejected, providing a high fidelity measurement of angular displacement. It will be appreciated that while such an angle sensor strip 14 provides an accurate angular displacement, the location of the bend can be anywhere along the angle sensor strip 14.

In embodiments, the upstanding teeth-like elements 16 are mounted to the angle sensor strip 14. Conductive strips, plates, or conductors 18 are disposed between the teeth 16 (or between certain teeth) that, when the sensor strip 10 is in a flat state (see FIG. 9A), are separated or non-contacting, and when the sensor strip 10 is in a bent upward state (see FIG. 9B and the arrows at 20) the conductors 18 are in contact with one another. This is a normally open contact arrangement. It will be appreciated that when the sensor strip 10 is bent in a downward direction opposite to that shown in FIG. 9B (opposite of the arrow at 20), the sensor strip 10 will be in the non-contacting state.

The teeth 16 in the sensor strip 10 shown in FIGS. 10A and 10B are rectangular (as opposed to trapezoidal) and are in an opposite configuration to that of the strip 10 in FIGS. 9A and 9B. That is, the straight sensor strip 10 of FIG. 10A is a normally closed arrangement that, when the sensor strip 10 is flat, is in the contact state and when the sensor strip 10 is bent in a downward direction (as indicated by the arrow at 21) in is in non-contact state. The sensor strips 10 can indicate when there is a bend in the sensor strip 10 and the location of the bend.

Thus, the combination of the teeth 16 which sense the location of a bend mounted to the angle sensor strip 14 which accurately measures the extent (angle) of the bend provides an accurate indication of the location and extent of a bend in the sensor 10. In embodiments, the teeth-like elements 16 can be mounted to the angle sensor strip 14 or to a member (such as a silicone strip) that is mounted to the angle sensor strip 14.

In an embodiment, the combination of the teeth 16 and the angle sensor strip 14 facilitates detecting the location of the bend, which is utilized in a mathematical model and the bend angle. Sensors 10 can be placed together in a chain to provide additional information regarding the shape of the surface.

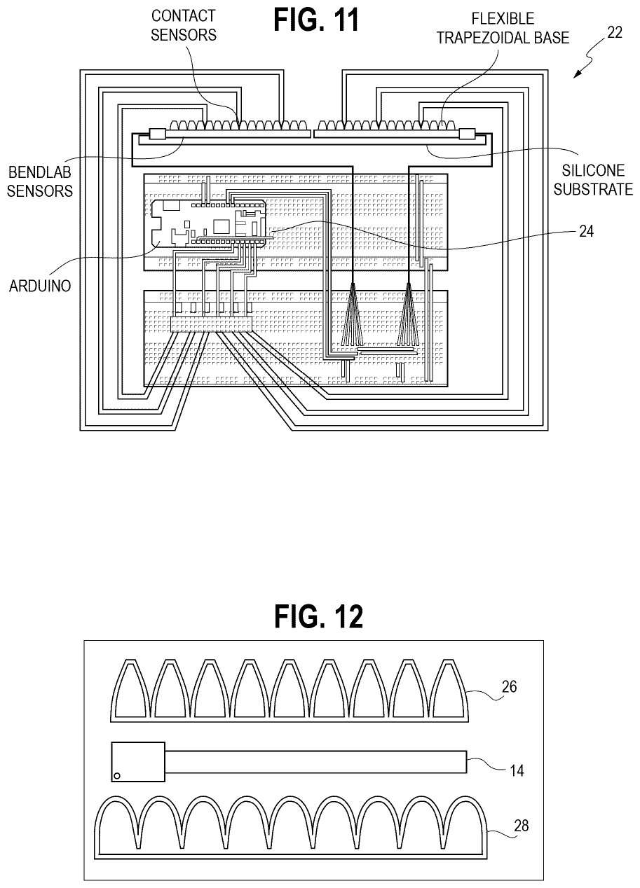

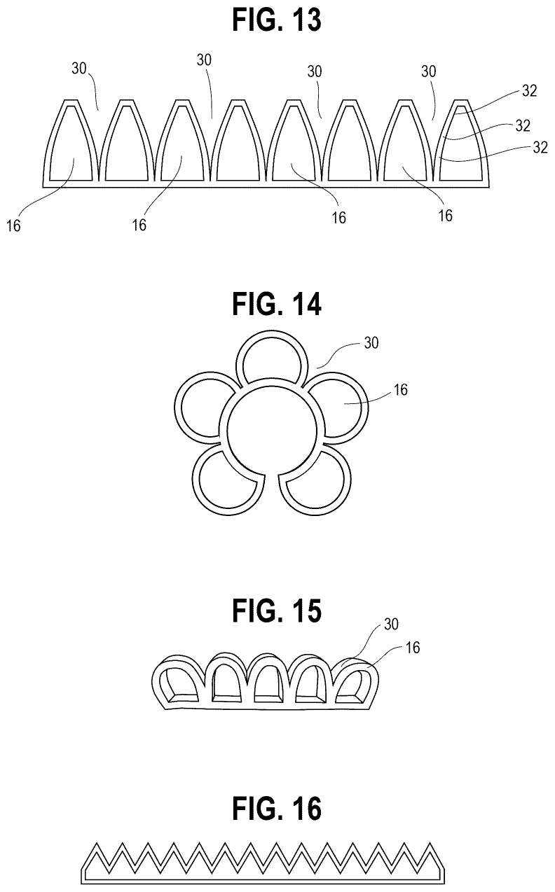

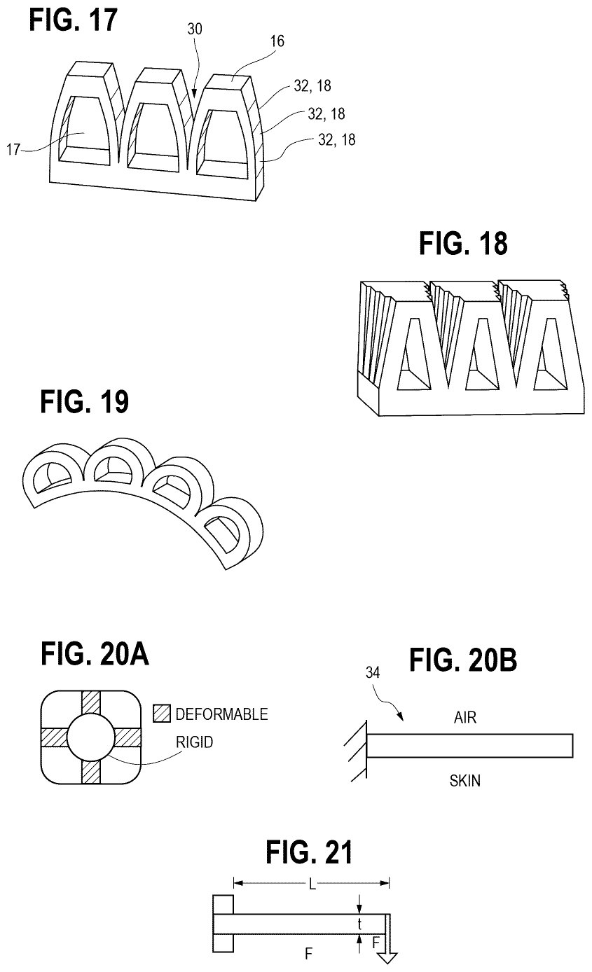

FIG. 11 is an embodiment of a prototype sensor system 22 and illustrates circuitry 24 that is configured to capture the data obtained by the sensor strip 10, which is the data captured by the angle sensor strip 14 and the conductors 18 between the teeth 16. FIG. 12 illustrates an embodiment of an angle sensor strip 14 sandwiched between a normally open toothed sensor strip 26 and a normally closed toothed sensor strip 28. FIG. 13 illustrates an embodiment of a toothed strip that has seven sensing regions 30 (the spaces between teeth 16) in which each region 30 has three angle sensing locations 32 (indicated by the conductors 18). FIGS. 14-17 illustrate various embodiments of teeth 16, FIG. 18 illustrates an embodiment in which a normally open sensor 26 has two sensing regions 30 with angle locations in two axes, and FIG. 19 illustrates a normally closed sensor 28 with three sensing regions 30. As illustrated in FIG. 17, the center 17 of the teeth 16 an be open.

The chained resistive flex sensors 10 were used to predict curvature based on a cantilever bending model 34 as shown in FIGS. 20A and 20B. This model 34 was used to relate experimental data to mathematical equations to determine the model for chaining multiple sensors together. The selection of the cantilever bending model stemmed from the assumption that one end of the flex sensor would remain fixed and the remainder of the flex sensor beyond the sensor end would deform or bend. The cantilever equation, Eq. 1, was used for the predictive modeling and subsequent validation of the modeling, as illustrated in FIG. 21.

$$δ = F {L}^{3} / 3 EI \qquad Eq . 1$$

where δ is the deflection,
F is the force,
L is the length of the beam,
E is the modulus of elasticity, and
I is the moment of inertia

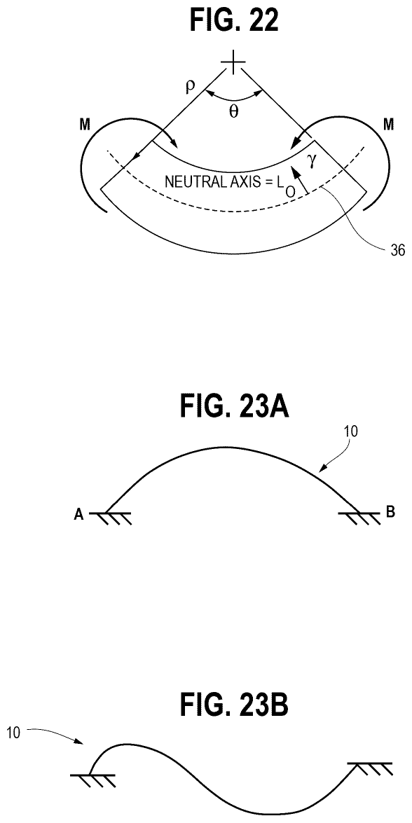

Referring to FIG. 22, the radius of the sensor 10 can be estimated at a certain distance from the neutral axis 36 by using the strain as shown in equation Eq. 2, below, in which ρ represents the radius at that point in the beam and y represents the neutral axis.

$$ε = - y / ρ \qquad Eq . 2$$

where ε is the strain,
y is the distance from the neutral axis to the top/bottom of the beam (½ of the thickness of the beam), and
ρ is the distance measured along the radius of curvature (θ) to the neutral axis.

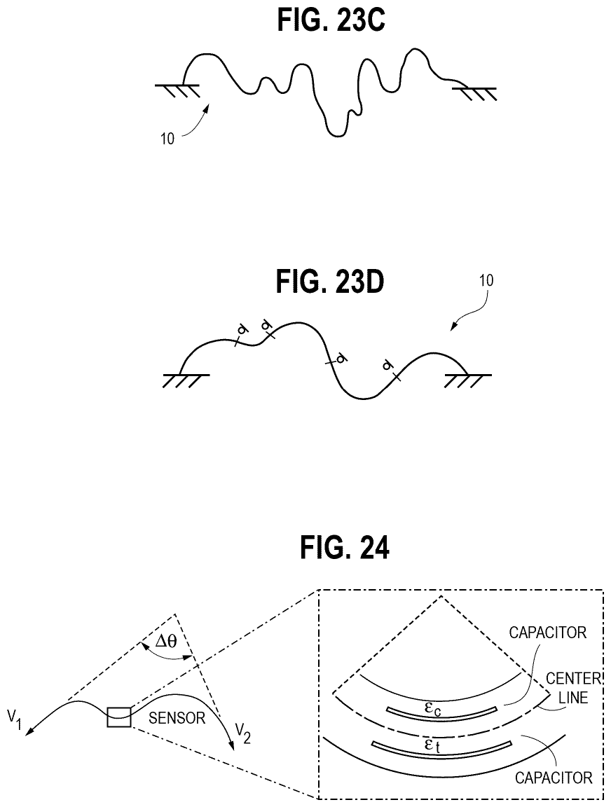

Sensors 10 were chained end-to-end in order to predict more complex shapes such as those illustrated in FIGS. 23B-23D. Using this model as implemented on a barrier, the flex sensors would be small enough so that each sensor 10 would only bend as a simple shape (a single bend) that could be predicted by the cantilever model (see, e.g., FIG. 23D). In this arrangement, the sensor 10 would display the same voltage value when bent in different shapes as shown in FIG. 23D. By implementing this constraint, the shape of the sensor 10 can be predicted through the cantilever beam model.

In such a model it was also assumed that the connections between sensors 10 would always be tangent (i.e., points of inflection) as shown in FIG. 23D, which ensures that the sensor 10 chain could predict the shape of a curve without concern of sharp corners between sensors 10.

As noted above, the BendLabs angle sensor strip 14 was tested due to its robust codebase for sensor characterization and data acquisition. These angle sensor strips 14 are flexible and have a Young's Modulus of 360 MPa which meets the specification for flexibility. Further, these angle sensor strips 14 are capacitive rather than resistive, they are sufficiently flexible, can detect in-plane tension, and have well-documented mechanical properties.

The capacitors stretch as the angle sensor strip 14 bend stretches which creates a differential capacitance that can be correlated with an angle measurement as shown in FIG. 24. The angle sensor strip 14 material is a medical grade silicone elastomer with conductive fillers which gives it similar mechanical properties to other silicone elastomer products. As such, the angle sensor strip 4 should interfere less with in-plane tension (stretching).

Advantageously, there is also a linear relationship between strain and capacitance. The strain of the angle sensor strip 14 is related to the capacitance when the angle sensor strip 14 is stretched or bent divided by the initial capacitance. The output angle is determined by integrating the curvature along the length of the angle sensor strip 14, which can be useful in working back from the angle to find stress and strain along the sensor. One property that we also found is that the angle sensor strip 14 has a Poisson's ratio of 0.5, meaning that it is an incompressible material that is deformed elastically at small strains.

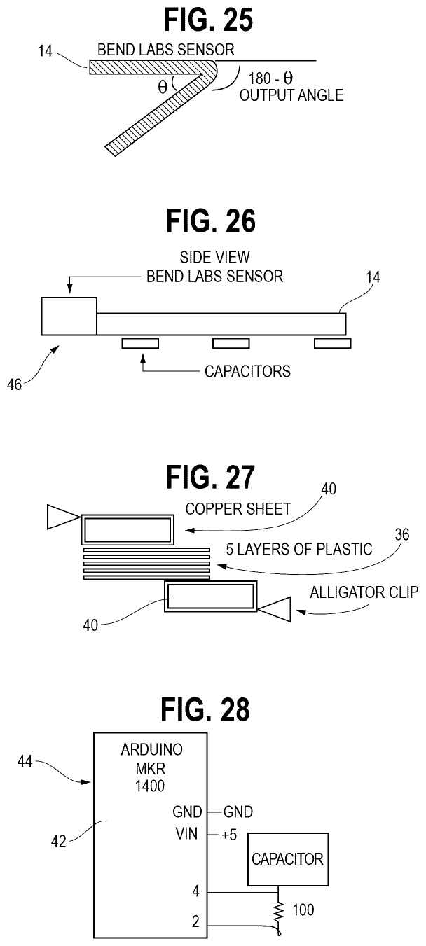

As noted above, the angle sensor strip 14 measures angle and percentage strain, however, it is only able to provide one angle measurement for the entire length of the angle sensor strip 14. As such, the final angle reading is a sum of all of the angles along the angle sensor strip 14. Additionally, the angle that the angle sensor strip 14 outputs is the supplementary angle to the angle at which it is bent, or the supplementary angle of the object around which it is wrapped. For example, as illustrated in FIG. 25, if the angle sensor strip 14 is wrapped around a 60° angle object, it outputs 120′. Further, it was noted that such an angle sensor strip 14 sensor was incompatible with the predictive cantilever model in that the angle sensor strip 14 was not sufficiently rigid. Other types of capacitance sensors were likewise evaluated for use in the skin barrier sensor.

Capacitive sensing technology was explored with the concept that it could be used in combination with the BendLabs sensor 14 to detect the location of bends as shown in FIG. 26. To create these capacitive sensors 46, five sheets of polyimide 38 were used as a dielectric material sandwiched between two plates of copper foil paper 40. The copper sheets 40 were placed at an offset from each other for ease of connection. The construction of the capacitive sensors 46 is shown in FIG. 27. The sensor 46 was then connected to a board 42 (in this arrangement, an Arduino MKR 1400 was used as shown in the circuit diagram 44 in FIG. 28) and the analog voltage values were read.

The capacitive sensor 46 could detect changes in the capacitance by applying a force on the sensor 46, and was found to detect small changes in capacitance without applying a large amount of pressure. Such a sensor 46, however, was unable to detect bending.

Based on the capacitive sensor 46, a more comprehensive sensor 10 was constructed. A series of flexible trapezoidal base tooth-shaped elements 16 were created using a 3D printer. Copper plate contact conductors 18 were positioned between the teeth 16 of the structure and connected to the board 42 (the Arduino) to detect when the conductors 18 were and were not in contact with one another, referring back to FIGS. 9A and 9B. In this manner, the sensor 10 detects the location of the bend.

The trapezoidal base teeth 16 were mounted to a BendLabs strip sensor 14 which was also connected to the Arduino 42 to provide a reading of the angle of bend. The sensors 10 were placed together in a chain to provide additional information regarding the shape of the surface S.

A base or substrate 12, such as the rubber silicone, was used as a surface onto which the strip sensor 14 and teeth 16 were attached. The substrate 12 provides a stable structure for the various sub-components. The silicone substrate 12 is sufficiently deformable (bendable) to deform into the shape of a curved surface S to be measured. One suitable silicone rubber substrate is a DRRAGON SKIN™ high performance silicone rubber. A suitable adhesive, such as a silicone adhesive was used to mount the sensor to the substrate.

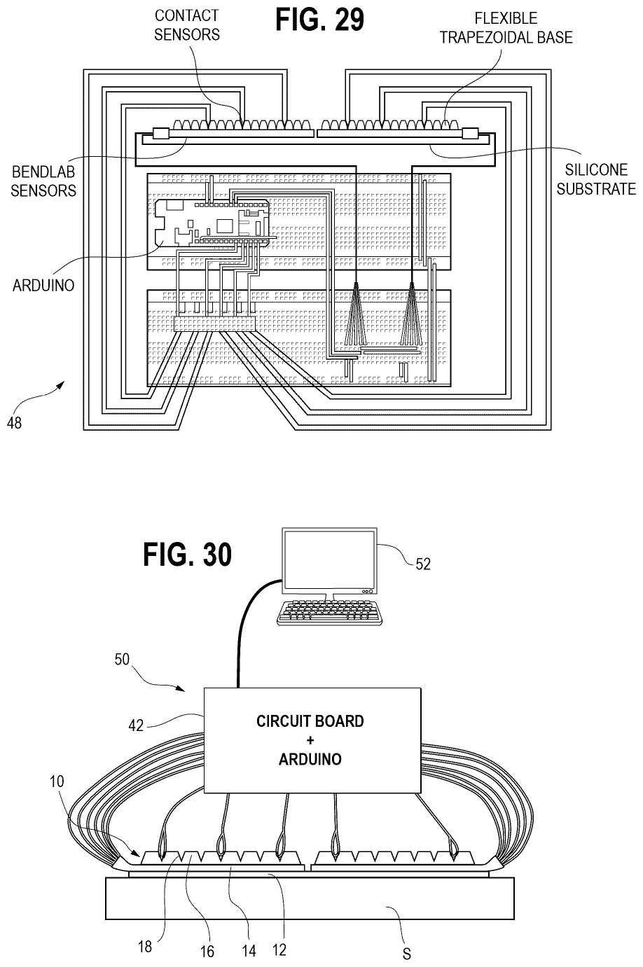

FIG. 29 shows the electrical architecture 48, and FIG. 30 shows the product architecture 50 including a computer 52, the board 42 (and Arduino), as well as the surface S or item to be monitored, the substrate 12, the BendLabs sensor 14, the trapezoidal base tooth-shaped elements 16, and all interconnections. The circuit board 42 was connected to the computer 52 where a heat map of the different stresses and strains sustained by each sensor 10 was visualized.

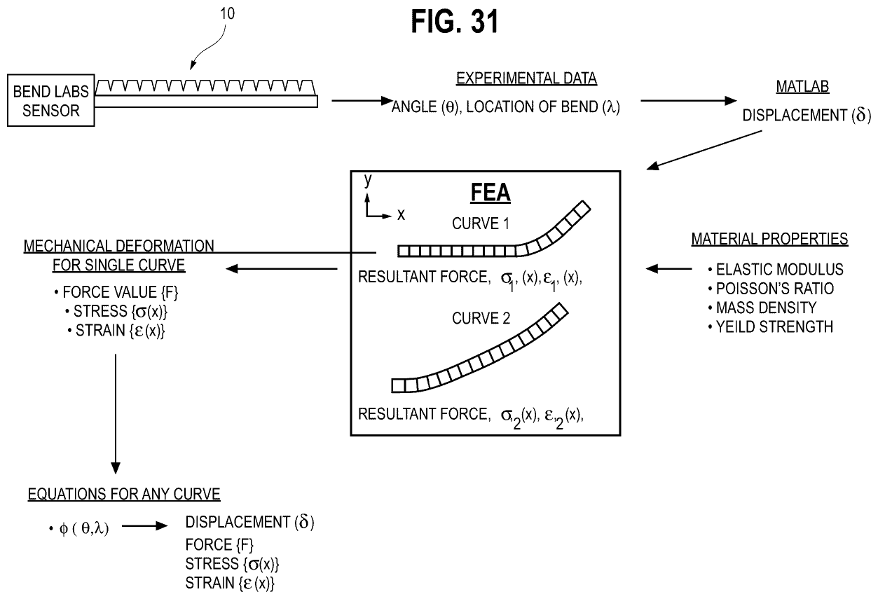

FIG. 31 is a workflow diagram showing how the data collected was used to create a predictive model. The BendLabs strip sensor 14 reports the supplementary angle to the angle of bend and the conductors 18 on the teeth 16 report the location of that bend along the length of that sensor 10. These two values are given to a displacement equation in a programing and numeric computing platform, such as MATLAB® to find the corresponding displacement value for that angle and location of the bend.

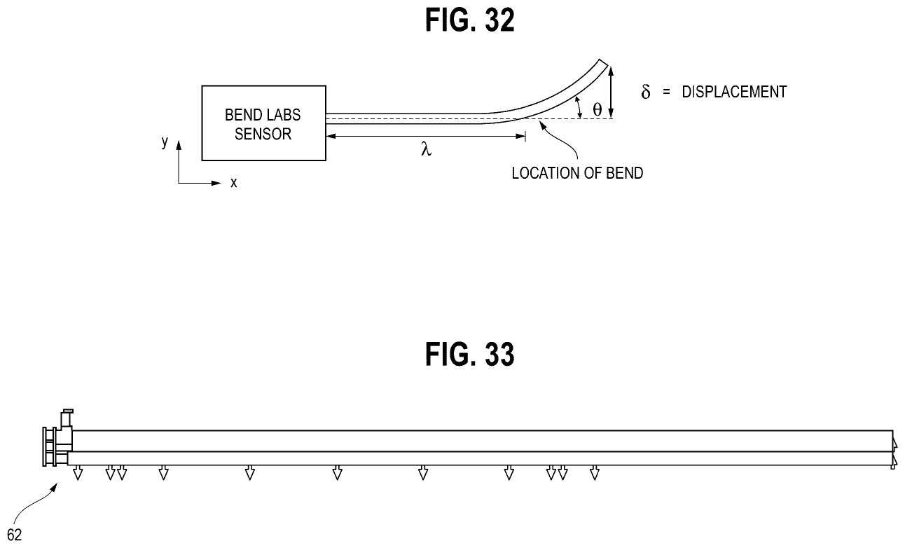

Finite element analysis was then used to make a model of the BendLabs sensor 12 and to input the calculated displacement from the MATLAB® model as a prescribed displacement to find the corresponding force and the stress and strain along the length of the sensor 10. Multiple cases of this simulation were run to build a predictive model from which an angle and location of bend could be used to provide the corresponding displacement, force, stress, and strain. FIG. 32 shows the method that was used to find the corresponding displacement to an angle and distance from the end of the sensor. A trigonometric relationship was used to obtain the relationship between the location of bend (λ), the displacement (δ), and the angle of bend (θ). The relationship is shown in equation 3, below.

$$Displacement ( θ ) = F ( θ , λ ) = sin ( θ ) ( 0.1 - λ ) \qquad Eq . 3$$

The displacement and location information was then used along with finite element analysis (FEA) in a CAD platform, such as SOLIDWORKS® to find a relationship between the location of bend, angle of bend, and the corresponding force. A model of the BendLabs sensor 12 was created with the mechanical properties of the BendLabs sensor 12 shown in Table 1, below.

TABLE 1
BendLabs Sensor Properties

| Property | Value | |
|---|---|---|
| Young's Modulus | 3600000 | MPa |
| Poisson's Ratio | 0.49 | |
| Mass Density | 1200 | kg/m\hat{ }3 |
| Yield Strength | 7901392 | N/m\hat{ }2 |

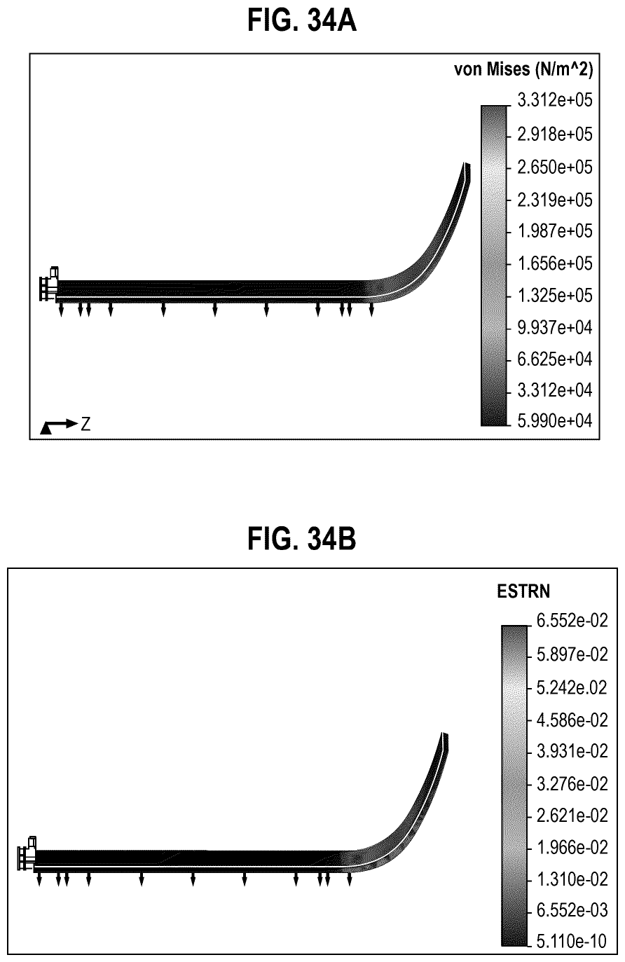

The model was established as follows: there was a fixed relationship on one of the end of the sensor 10, a fixed relationship along the part of the sensor 10 that was not bending and a prescribed displacement along the other edge of the sensor 10 as shown in FIG. 33. A sample of the FEA results showing the corresponding stress and strain plots for the model is shown in FIGS. 34A and 34B, in which FIG. 34A shows the FEA results for stress and FIG. 34B shows the FEA results for strain.

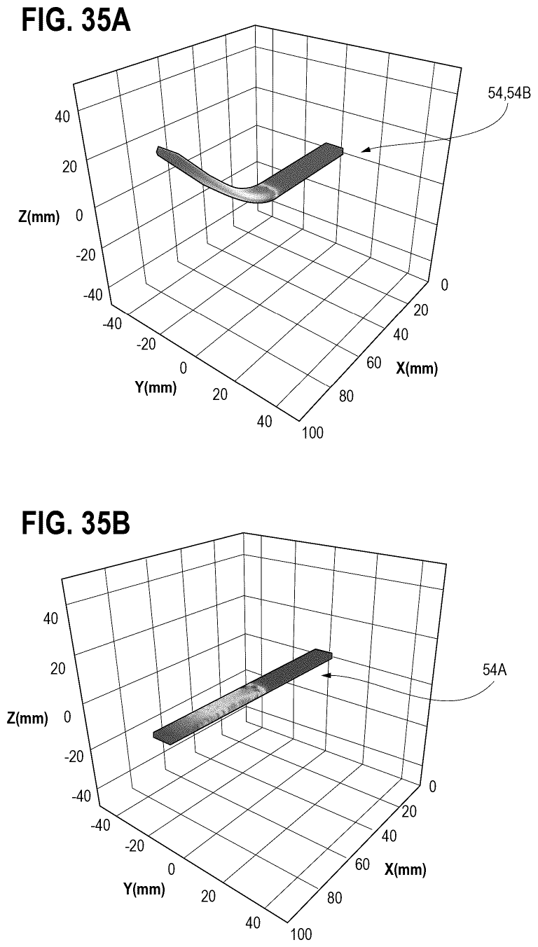

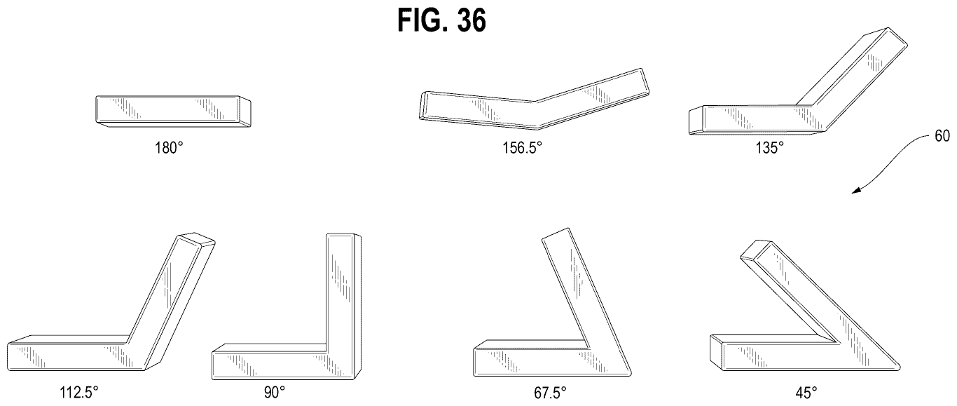

Referring to FIG. 35, a heatmap 54 was used to convey displacement, stress, and strain data collected by the sensors 10. The heat map 54 allows for an understanding of the distribution of the induced deformation, stress, and strain as the sensor 10 distorts. Using deformation data exported from FEA, these visualizations could be generated. Strain, stress, and displacement can all be determined for each node of the geometry of the sensor 10, which is assumed and represented as an isotropic elastic beam.

A heat map containing the approximate spatial distribution of the sensor geometry and one of the aforementioned FEA-derived output variables as a fourth dimension are possible in two different kinds of modes: a static heat map 56 that describes the distribution of the stress, strain, or displacement while leaving the sensor geometry undistorted; or a ‘dynamic’ heat map 58 that manifests a distorted sensor geometry as a result of the induced deformation (from the resultant applied force indicated in the FEA software). Both can be implemented in a simple manner; the only difference is in choosing to include the resultant displacement of the sensor 10 while visualizing the stress (or strain) distribution. The dynamic heat map 58, as illustrated in FIG. 35A, may be more insightful given that it allows for observing the relationship between the displacements and stresses in the same graph. The static heatmap 56 method is illustrated in FIG. 35B.

Referring to FIG. 36, several angle test fixtures 60 were created for testing to determine whether the angle measured by the BendLabs sensor 12 was accurate and whether that angle combined with the information provided by the conductors 18 between the teeth 16 on bend location would be able to accurately predict the displacement. The sensors 10 were placed along the angle fixture and test results were obtained through the values reported by the MATLAB® code. The independent variables were angle and displacement and the dependent variable was displacement.

Table 3, below, shows two initial tests that were performed to determine the system's performance. Test number 1 was run to determine whether the BendLabs sensor 12 could accurately measure an angle and whether the conductors 18 between the teeth 16 could reliably give the bend location. It was found that there was a significant amount of noise in the sensor 10. Test number 2 was run to calculate the expected displacement given an angle and location of bend and placed a bend of that angle at that location to determine whether the sensor 10 would accurately give the magnitude of displacement. Similar to test 1, there was noise in the sensor 10 and the sensitivity to bends at the edge of the sensor 10. Again, it is envisioned that filters can be used to reduce or eliminate the noise.

TABLE 2
Test Results for Initial System Performance Tests

| | | |
|---|---|---|
| Expected Value | Tested Value | % Error |

Test #1- Angle Measurement and Location

| | | |
|---|---|---|
| 45 degree angle | 44.6 degree angle | 0.88% |
| 51 mm location | 51 mm location | 0% |
| 22.5 degree angle | 19.6 degree angle | 12.9% |
| 70 mm location | 69 mm location | 1.43% |

Test #2- Displacement Test

| | | | | |
|---|---|---|---|---|
| 35 | mm | 37.06 | mm | 5.89% |
| 19.13 | mm | 17.5 | mm | 8.52% |

Overall, testing showed three principal sources of error: (1) the sensor's 10 ability to accurately measure an angle depended on how it was calibrated; (2) the edge 62 of the sensor 10 was not adhered to the silicone substrate 12 which made it bend differently from the rest of the sensor 10; and (3) noise. The first and second error sources are readily addressed by: (1) proper calibrations of the strip sensor 14; and (2) affixing the entirety of the strip sensor 14 to the substrate 12. As noted above, the error associated with noise can be addressed by the use of filters.

Embodiments of a peristomal skin barrier sensor 10 include a normally-open contact sensor 26 made of a flexible material that includes three or more sensing regions 30 having sensor teeth 16, with conductors 18 placed at a distinct angle between the sensor teeth 16. One suitable angle is 20°. The number of sensing regions 30 can vary to allow for more or less data collection along the length of the sensor 10. The angles and number of angles can vary to allow for more or less angle measurements along the length of the sensor 10. The size and/or shape of the conductors 18 between the teeth 16 can likewise vary.

Embodiments may also include a normally-closed contact sensor 28 made of a flexible material that includes three or more sensing regions 30 having sensor teeth 16, with conductors 18 placed at a distinct angle between the sensor teeth 16. One suitable angle is 20°. The number of sensing regions 30 can vary to allow for more or less data collection along the length of the sensor 10. The angles and number of angles can vary to allow for more or less angle measurements along the length of the sensor 10. The size and/or shape of the conductors 18 between the teeth 16 can likewise vary.

In embodiments, the peristomal skin barrier sensor 10 can include a normally-open contact sensor 26 made of thermoplastic polyurethane which includes seven sensing regions 30 with conductors 18 placed at more than one distinct angle between the sensor teeth 16. The angles can be, for example, 5°, 10°, and/or 15°. In embodiments, the material selected is another suitable thermoplastic elastomer. In embodiments, the number of sensing regions 30 can vary to allow for more or less data collection along the length of the sensor 10. The number of angles can also vary to allow for more or less angle measurements, and the size and/or shape of the conductors 18 can likewise vary.

In embodiments, the normally-closed contact sensor 28 can include two sensing regions 30 with conductors 18 placed at two distinct angles between the sensor teeth 16. Suitable angles can be, for example, 5° and 10°. The number of sensing regions 30 can vary to allow for more or less data collection along the length of the sensor 10. The number of angle may also vary to allow for more or less angle measurements.

It is envisioned that the sensor 10 can be used to create a barrier with a dynamic response system that counteracts any lifting that occurs or specializes in custom barriers to better fit a patient's abdomen topology. Such barriers can facilitate an ostomy barrier and pouch that is more securely adhered to the user and minimizes discomfort and leakage.

In the present disclosure the words “a” or “an” are to be taken to include both the singular and the plural. Conversely, any reference to plural items shall, where appropriate, include the singular. The words “first,” “second,” “third,” and the like may be used in the present disclosure to describe various information, such information should not be limited to these words. These words are only used to distinguish one category of information from another. The directional words such as, but not limited to, “upper” “lower” “raised” “lowered” “top” “bottom” “above” “below” “up” “down” “alongside” “front” “back” “left” and “right” and the like are used for purposes of illustration and as such, are not limiting. Depending on the context, the word “if” as used herein may be interpreted as “when” or “upon” or “in response to determining.”

From the foregoing it will be observed that numerous modifications and variations can be effectuated without departing from the true spirit and scope of the novel concepts of the present disclosure. It is to be understood that no limitation with respect to the specific embodiments illustrated is intended or should be inferred. The disclosure is intended to cover by the appended claims all such modifications as fall within the scope of the claims.

What is claimed is:

1. A peristomal skin barrier sensor comprising:
a flexible substrate;
a flexible angle sensor strip positioned on the substrate;
a series of teeth-like projections positioned on the flexible angle sensor strip; and
conductors mounted on a portion of at least some of teeth-like projections of the series of teeth-like projections,
wherein the flexible angle sensor and the conductors are in electrical communication with a processor, and
wherein a location of a bend in the sensor is identified via electrical signals from the conductors and an angle of the bend in the sensor is identified via electrical signals from the flexible angle sensor.

2. The sensor of claim 1, wherein the series of teeth-like projections and the conductors form a normally open sensor.

3. The sensor of claim 2, wherein the teeth-like projections are trapezoidal shaped teeth and wherein the conductor are mounted on an angled surface of the trapezoidal shaped teeth.

4. The sensor of claim 3, wherein sides of the trapezoidal shaped teeth-like projections extend upwardly at an angle relative to horizontal axis through the teeth.

5. The sensor of claim 4, wherein the angle is about 20 degrees.

6. The sensor of claim 1, wherein the teeth have an open interior.

7. The sensor of claim 2, wherein the teeth-like projections have a first upwardly extending surface at a first angle relative to horizontal axis through the teeth-like projections and a second upwardly extending surface extending from the first upwardly extending surface at a second angle relative to the horizontal axis, and wherein the first angle is less than the second angle.

8. The sensor of claim 7, wherein the first angle is about 5 degrees to about 10 degrees and wherein the second angle is about 10 degrees to about 20 degrees.

9. The sensor of claim 7, wherein a first conductor is positioned on the first surface and a second different conductor is positioned on the second surface.

10. The sensor of claim 1, including a plurality of series of teeth-like projections, and wherein the conductors on each of the plurality of series of teeth-like projections is electrically isolated from the conductors on the others of the plurality of series of teeth-like projections.

11. The sensor of claim 1, wherein the series of teeth-like projections and the conductors form a normally closed sensor.

12. The sensor of claim 11, wherein the teeth-like projections have a round profile.

13. A peristomal skin barrier sensor comprising:
a skin barrier, wherein the skin barrier surrounds a stoma that emits ostomy dejecta; and
a skin barrier sensor configured to a sense movement of the skin barrier, the skin barrier sensor having flexible substrate, a flexible angle sensor strip positioned on the substrate, a series of teeth-like projections positioned on the flexible angle sensor strip, and conductors mounted on a portion of at least some of teeth-like projections of the series of teeth-like projections,
wherein the flexible angle sensor and the conductors are in electrical communication with a processor, and
wherein a location of a bend in the sensor is identified via electrical signals from the conductors and an angle of the bend in the sensor is identified via electrical signals from the flexible angle sensor.
